(19) United States
(12) Patent Application Publication
(10) Pub. No.: US 2009/0088756 A1
(43) Pub. Date: Apr. 2, 2009

(54) SYSTEM AND METHOD FOR SECURING SURGICAL IMPLANT

(75) Inventor: Peter Anderson, Andover, MA (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/865,309

(22) Filed: Oct. 1, 2007

(51) Int. Cl. A61B 17/00; A61B 5/05

(52) U.S. Cl. 606/80; 600/409; 600/424

(57) ABSTRACT

In one embodiment, a system for locating a transverse hole disposed at a predetermined location in a surgical implant inserted into a fractured bone of a patient and for drilling the fractured bone coaxially with the transverse hole is provided. The system comprises a first electromagnetic sensor unit coupled to the surgical implant, a surgical instrument for drilling a hole through the fractured bone coaxially with the transverse hole, the surgical instrument having a main axis disposed in parallel to the axis of the transverse hole, an electromagnetic drive unit coupled to the surgical instrument for energizing the first electromagnetic sensor unit to transmit a signal, a second electromagnetic sensor unit configured for receiving the signal and a display unit for converting the signal into visual signals enabling a user to change the position of the surgical instrument to align the surgical instrument with the transverse hole of the surgical implant.

### FIELD OF THE INVENTION

The invention relates generally to the field of surgery, and in particular relates to techniques for aligning a surgical instrument with holes of a surgical implant.

### BACKGROUND OF THE INVENTION

A current surgical treatment for fractures of the shaft of long bones (e.g., femur and tibia) is the insertion of an intramedullary rod (IM rod). The IM rods are relatively rigid devices inserted into one end of the bone and down the center canal of the bone shaft, such that the fracture site is bridged. Transverse holes in either end of the IM rod receive screws inserted transversely through the bone in order to lock the two bone fragments relative to one another. The insertion of the screws farthest from the IM rod insertion hole is currently a difficult and time consuming procedure requiring numerous x-ray images. An intraoperative x-ray machine (C-arm) is repeatedly fired and reoriented until it is exactly aligned with the transverse holes as evidenced by images displaying the holes as “perfect circles”. To establish a starting point, a surgeon uses further x-ray images to align the drill tip with the images of the holes. The surgeon then uses the source-to-receiver axis of the C-arm as an external reference frame along which the long axis of the drill is oriented. Even after this, several attempts may be required to drill the holes into the bone and through the transverse holes.

Several alternative approaches have been employed in an attempt to speed this process. External jigs have been tried with little success as inaccuracies in the jig leads to inaccuracy of the mounting between the jig and the IM rod, and deformations of the IM rod accumulate to cause the final jig hole positions to be unreliably aligned with the IM rod holes. Radiolucent drills and surgical instruments and laser sighting devices have been developed which, in the best cases, improve the speed and accuracy of hole placement. However these techniques still require a significant number of x-ray images to be obtained in order to first achieve a C-arm orientation that produces “perfect circles” in the images.

Hence there exists a need for a system and method for efficiently and accurately locating a target point in a surgical implant.

### BRIEF DESCRIPTION OF THE INVENTION

The above-mentioned needs are addressed and can be understood by reading and understanding the subject matter described herein. Various other features, objects, and advantages of the subject matter described herein will be made apparent to those skilled in the art from the accompanying drawings and detailed description.

In one embodiment, a system for locating a transverse hole disposed at a predetermined location in a surgical implant inserted into a fractured bone of a patient and for drilling the fractured bone coaxially with the transverse hole is provided. The system comprises a first electromagnetic sensor unit coupled to the surgical implant, a surgical instrument for drilling a hole through the fractured bone coaxially with the transverse hole, an electromagnetic drive unit coupled to the surgical instrument for energizing the first electromagnetic sensor unit to transmit a signal, a second electromagnetic sensor unit configured for receiving the signal and a display unit coupled to the first and second electromagnetic sensor units for converting the signal into visual signals enabling a user to change the position of the surgical instrument to align the surgical instrument with the transverse hole of the surgical implant.

In another embodiment, a method of aligning a surgical instrument with a transverse hole of a surgical implant, the surgical implant being insertable into a patient's bone and having a hollow body portion, is provided. The method comprises steps of inserting a first electromagnetic sensor unit capable of emitting a signal within the hollow body portion of the surgical implant such that the signal passes through the transverse hole of the surgical implant, exciting the first electromagnetic sensor unit with a electromagnetic drive unit to cause the first electromagnetic sensor unit to emit the signal, the electromagnetic drive unit coupled to the surgical instrument, tracking the signal emitted by the first electromagnetic sensor unit using a second electromagnetic sensor unit, the signal comprising position and orientation information of the transverse hole of the surgical implant, displaying the position and orientation of the transverse hole of the surgical implant, tracking the position and trajectory of the surgical instrument, displaying the position and trajectory of the surgical instrument, superimposing the position and orientation of the transverse hole of the surgical implant and the position and trajectory of the surgical instrument on at least one image of the patient and aligning the surgical instrument with the position and orientation of the transverse hole.

In yet another embodiment, a method of securing a surgical implant into a bone of a patient is provided. The method comprises steps of placing a first electromagnetic sensor unit in the surgical implant, the surgical implant having at least one transverse hole for receiving a connector to secure the surgical implant to the bone, inserting the surgical implant into the bone, placing a second electromagnetic sensor unit external to the body of the patient, placing an electromagnetic drive unit carried in a fixed position by a surgical instrument, determining the position and orientation of the surgical instrument relative to the transverse hole, aligning the surgical instrument with the transverse hole by viewing a virtual representation of the position and orientation of the transverse hole and the surgical instrument as indicated on a display unit and drilling a hole in the bone in alignment with the transverse hole.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 is a schematic diagram representing a fractured bone of a patient;

FIG. 2 is a block diagram of an embodiment of a system for locating a transverse hole in an embodiment of the invention;

FIG. 3 shows a schematic diagram representing a location of a first electromagnetic sensor unit and a second electromagnetic sensor unit in an exemplary embodiment of the invention;

FIG. 4 shows a schematic representation of the system for locating a transverse hole shown in FIG. 2;

FIG. 5 is a flow diagram depicting an embodiment of a method of aligning a surgical instrument with a transverse hole of a surgical implant;

FIG. 6 is a flow diagram depicting an embodiment of a method of securing a surgical implant into a bone of a patient; and

FIG. 7 is a flow diagram depicting an embodiment of a method of determining the position and orientation of a surgical instrument.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the invention. The following detailed description is, therefore, not to be taken in a limiting sense.

An embodiment of the invention provides a system and method for locating an electromagnetic sensor unit placed within a surgical implant, such as an intramedullary rod, used in orthopaedic surgery.

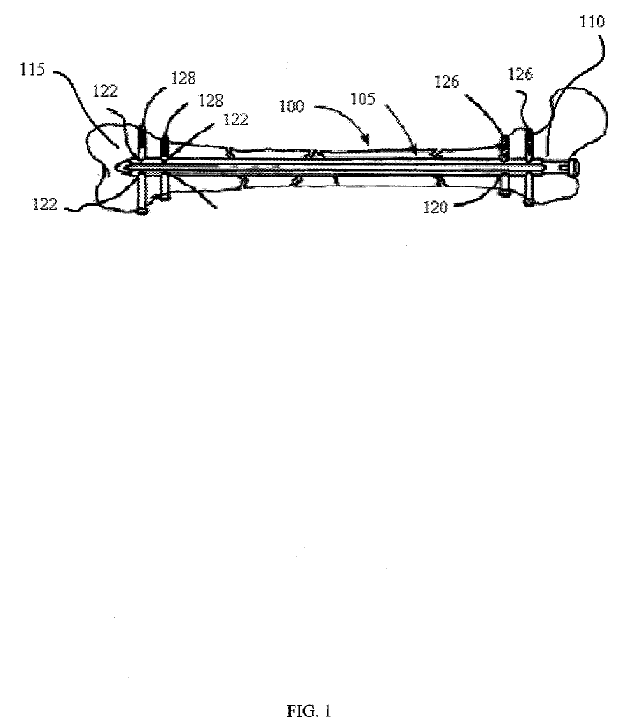

In FIG. 1, a broken long bone 100, for example, a broken femur, tibia, or humerus bone, is shown in which a conventional intramedullary rod 105 has been inserted into the interior region of the bone 100. The intramedullary rod 105 is an elongate metal rod having a hollow body portion with a proximal end 110 and a distal end 115. In one embodiment, the intramedullary rod 105, which is a particular type of surgical implant, is made of a metal such as titanium.

Further, the intramedullary rod 105 comprises a pair of transverse holes 120 and 122 at each of the proximal end 110 and the distal end 115. The intramedullary rod 105 is secured to the bone 100 by means of screws 126 and 128 or bolts (not shown in FIG. 1) which are installed transverse to the intramedullary rod 105 and which pass through the bone 100 at the transverse distal hole 120 and proximal hole 122 respectively, thereby securing the bone 100 and the intramedullary rod 105 to one another.

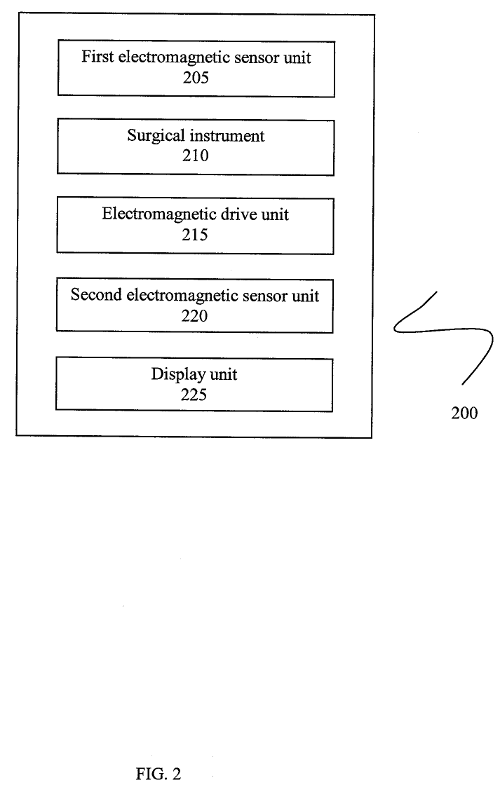

In one embodiment, the invention provides a system 200 for locating a transverse hole 120 or 122 disposed at a predetermined location in a surgical implant 105 inserted into a fractured bone 100 of a patient 305 and for drilling the fractured bone 100 coaxially with the transverse hole 120 or 122. The system 200 as shown in FIG. 2 comprises a first electromagnetic sensor unit 205 coupled to the surgical implant 105, a surgical instrument 210 for drilling a hole through the fractured bone 100 coaxially with the transverse hole 120 or 122, the surgical instrument 210 having a main axis disposed in parallel to the transverse hole 120 or 122, an electromagnetic drive unit 215 coupled to the surgical instrument 210 for energizing the first electromagnetic sensor unit 205 to transmit a signal, a second electromagnetic sensor unit 220 configured for receiving the signal and a display unit 225 coupled to the first and second electromagnetic sensor units 205 and 220 for converting the signal into visual signals enabling a user to change the position of the surgical instrument 210 in order to align the surgical instrument 210 with the transverse hole 120 or 122 of the surgical implant 105.

In one embodiment, each of the first electromagnetic sensor unit 205, the second electromagnetic sensor unit 220 or the electromagnetic drive unit 215 may include an optical sensor unit, an electro magnetic sensor unit, or any other sensing device or combination thereof operable to sense a changeable or variable position relative to one another and to generate an electrical output, such as a linear electrical output (LEO) or a digital electrical output (DEO), representative of the changeable or variable position. The electrical output of the first electromagnetic sensor unit 205 and/or the second electromagnetic sensor unit 220 can be expressed as, voltage potential, current, or other measurable electrical form.

The electromagnetic drive unit 215, the first electromagnetic sensor unit 205 and the second electromagnetic sensor unit 220 can each be a wireless sensor unit and may draw power from an external line source or may have a separate power source, such as a battery or a photocell, for example.

The first electromagnetic sensor unit 205 can be positioned on the surgical implant 105 so as to track the position of the transverse hole 122 in the surgical implant 105. Accordingly, the first electromagnetic sensor unit 205 can be coupled to the distal end 115 of the surgical implant 105. More specifically, the first electromagnetic sensor unit 205 can be placed inside the transverse hole 122 positioned at the distal end 115 of the surgical implant 105 such that the first electromagnetic sensor unit 205 is carried in a fixed position within the transverse hole 122 of the surgical implant 105.

In one embodiment, the second electromagnetic sensor unit 220 is placed external to the patient 305 such that, the second electromagnetic sensor unit 220 is placed in a fixed position relative to the transverse holes 120 and 122 of the surgical implant 105.

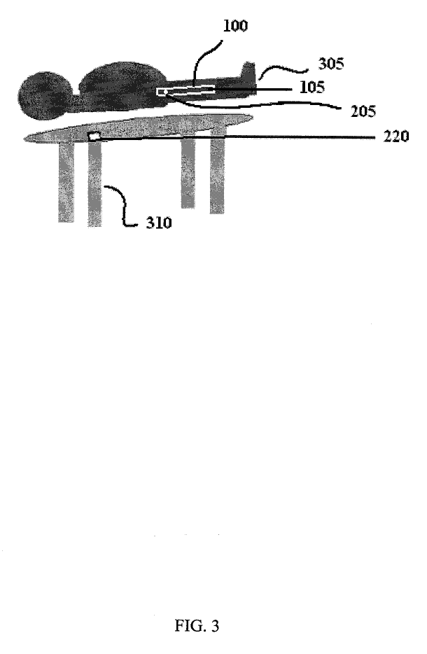

FIG. 3 shows a schematic diagram representing a location of the first electromagnetic sensor unit 205 and the second electromagnetic sensor unit 220. An exemplary embodiment of the system 200 comprises the first electromagnetic sensor unit 205 positioned on the surgical implant 105 inserted into the bone 100 of a patient 305, and the second electromagnetic sensor unit 220 located at a reference relative to movement of the first electromagnetic sensor unit 205. One embodiment of the reference includes a patient positioning assembly 310 supporting the patient 305. Yet, it should be understood that the reference is not limited to the above-mentioned examples and can vary (e.g., the floor or a wall of the room selected to provide the medical procedure, etc.).

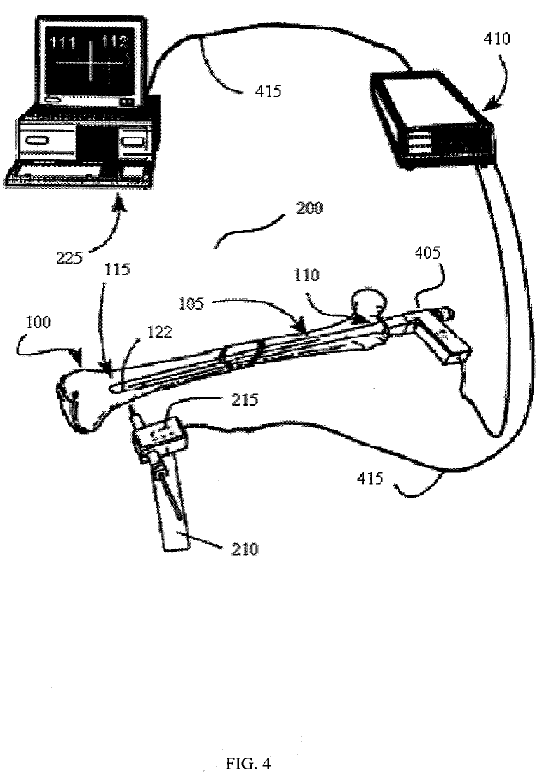

A schematic representation of the system 200 for locating the transverse holes 120 and 122 in the surgical implant 105 is shown at FIG. 4. A driver unit referred to hereafter as a surgical implant driver 405 may be used to insert the surgical implant 105 into the bone 100. The surgical implant driver 405 is comprised of a handle and longitudinal body. The longitudinal body has a longitudinal cylindrical bore with central axis, which is coincident with the longitudinal axis of the surgical implant 105 when attached securely to the surgical implant 105.

The electromagnetic drive unit 215 can be positioned external to the patient 305 such that the electromagnetic drive unit 215 is carried in a fixed position by the surgical instrument 210. The electromagnetic drive unit 215 comprises at least one transmitter coil capable of energizing the first electromagnetic sensor unit 205.

The first electromagnetic sensor unit 205 comprises at least one transceiver coil capable of emitting a signal responsive to the flux received from the electromagnetic drive unit 215, such that the signal is zero when the axis of the first electromagnetic sensor unit 205 is coplanar with the axis of the electromagnetic drive unit 215.

The second electromagnetic sensor unit 220 comprises at least one receiver coil capable of receiving the signal emitted by the first electromagnetic sensor unit 205. The second electromagnetic sensor unit 220 can be configured to detect, measure or sense a variable position such as an actual position and/or changes in position of the first electromagnetic sensor unit 205 relative to the electromagnetic drive unit 215 and to translate the detected or sensed variable position into a position data.

Accordingly, the second electromagnetic sensor unit 220 may include a processing unit 410. The signal received by the second electromagnetic sensor unit 220 corresponding to the position of the first electromagnetic sensor unit 205 and/or change in position of the first electromagnetic sensor unit 205 relative to the electromagnetic drive unit 215 may be fed to the processing unit 410. Signals received by the second electromagnetic sensor unit 220 are conveyed through a cable 415 to the processing unit 410. The processing unit 410 is configured to compute the position data based on the received signal.

The processing unit 410, by way of an algorithm, computes the position and orientation of the first electromagnetic sensor unit 205 with respect to the electromagnetic drive unit 215. The processing unit 410 then outputs the position and orientation information to the display unit 225, by which the surgeon can view the relative position of the surgical instrument 210 with respect to the surgical implant 105.

The display unit 225 may be configured to illustrate the position and trajectory of the surgical instrument 210 as well as the position and orientation of the surgical implant 105. The display unit 225 presents visual images that graphically indicate the manner in which the surgical instrument 210 is to be moved to bring the surgical instrument 210 into alignment with the transverse holes 120 and 122. Thus, the display unit 225 presents a continuous visual representation of the position of the surgical instrument 210 relative to the axis of the transverse hole 120 or 122 enabling the user to achieve a desired alignment.

In one embodiment, the processing unit 410 can be integrated with one of the second electromagnetic sensor unit 220 and the display unit 225. Alternatively, the processing unit 410 can be installed in an independent device. In yet another embodiment, the processing unit 410 can include software comprising a series of computer readable program instructions stored in a memory and operable to run on one of the second electromagnetic sensor unit 220, the display unit 225 or an independent device.

The position and orientation data of the transverse holes 120 and 122 in the surgical implant 105 and the position and orientation data of the surgical instrument 210 can be graphically displayed to the user so as to guide the user for accurate alignment of the surgical instrument 210 with the transverse holes 120 and 122 in the surgical implant 105. Moreover, the graphical display of the position of the surgical implant 105 and the surgical instrument 210 can be superimposed with at least one image of the patient 305.

The image of the patient 305 can be obtained using an imaging system such as a computed tomography (CT) system, a positron emission tomography (PET) system, a magnetic resonance (MR) imaging system, an ultrasound imaging system, or an X-ray imaging system. One of ordinary skill in the art shall however appreciate that the imaging system is not limited to the examples given above.

A superimposed display provides the position information of the surgical implant 105 and the surgical instrument 210 relative to the anatomy of the patient 305. By viewing a virtual representation of the surgical implant 105 and the surgical instrument 210 on the display unit 225, the surgical instrument 210 can be moved to a desired location relative to the surgical implant 105.

In one embodiment, the first electromagnetic sensor unit 205 installed in the surgical implant 105 can be remote from the transverse holes 120 and 122 so that the first electromagnetic sensor unit 205 may not be carried into the bone 100 along with the surgical implant 105. The processing unit 410 in the second electromagnetic sensor unit 220 calculates the offset from the first electromagnetic sensor unit 205 to the transverse holes 120 and 122, and thus, the display shows the position of the transverse holes 120 and 122 relative to the surgical instrument 210.

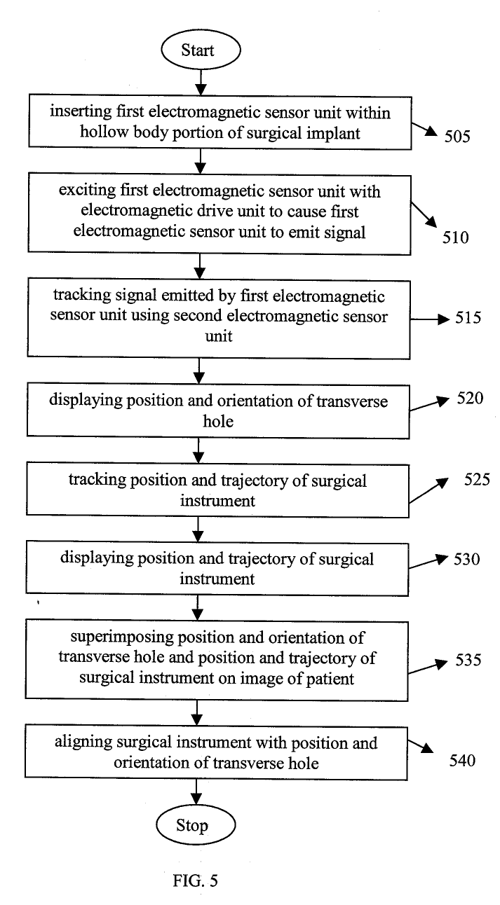

In another embodiment, as shown in FIG. 5, a method of aligning the surgical instrument 210 with the transverse hole 120 or 122 of the surgical implant 105 is provided. The method comprises steps of inserting the first electromagnetic sensor unit 205 capable of emitting a signal within the hollow body portion of the surgical implant 105 such that the signal passes through the transverse hole 122 of the surgical implant 105 step 505, exciting the first electromagnetic sensor unit 205 with the electromagnetic drive unit 215 to cause the first electromagnetic sensor unit 205 to emit a signal step 510, the electromagnetic drive unit 215 being coupled to the surgical instrument 210, tracking the signal emitted by the first electromagnetic sensor unit 205 using the second electromagnetic sensor unit 220 step 515, the signal comprising position and orientation information of the transverse hole 122 of the surgical implant 105, displaying the position and orientation of the transverse hole 122 of the surgical implant 105 in a display unit 225 step 520, tracking the position and trajectory of the surgical instrument 210 step 525, displaying the position and trajectory of the surgical instrument 210 step 530, superimposing the position and orientation of the transverse hole 122 of the surgical implant 105 and the position and trajectory of the surgical instrument 210 on at least one image of the patient 305 step 535 and aligning the surgical instrument 210 with the position and orientation of the transverse hole 122 step 540.

Alignment of the surgical instrument 210 to the axis passing through the transverse hole 122 of the surgical implant 105 aligns the surgical instrument 210 with the transverse hole 122, permitting accurate drilling of a hole though the bone 100 and the transverse hole 122 of the surgical implant 105.

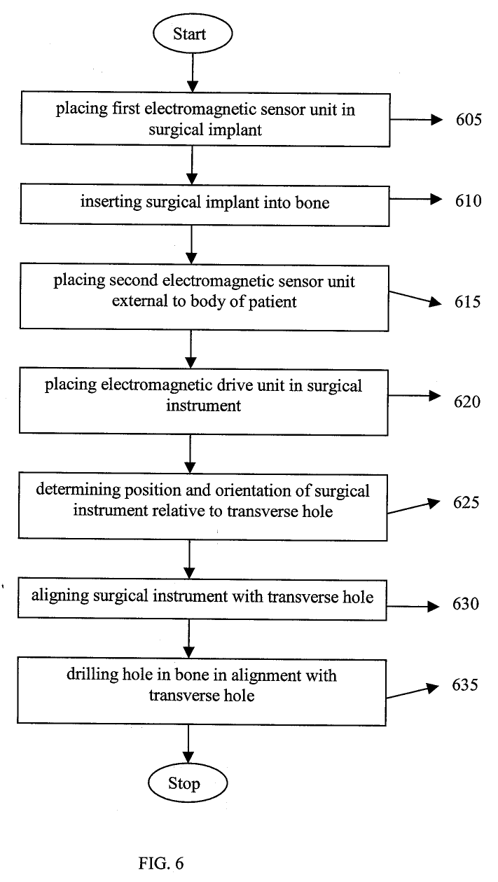

In yet another embodiment, as illustrated in FIG. 6, a method of securing the surgical implant 105 into the bone 100 of a patient 305 is provided. The method comprises steps of placing the first electromagnetic sensor unit 205 in the surgical implant 105 step 605, the surgical implant 105 having at least one transverse hole 120 or 122 for receiving a connector to secure the surgical implant 105 to the bone 100, inserting the surgical implant 105 into the bone 100 step 610, placing the second electromagnetic sensor unit 220 external to the body of the patient 305 step 615, placing the electromagnetic drive unit 215 carried in a fixed position by the surgical instrument 210 step 620, determining the position and orientation of the surgical instrument 210 relative to the transverse hole 120 or 122 step 625, aligning the surgical instrument 210 with the transverse hole 120 or 122 by viewing a virtual representation of the position and orientation of the transverse hole 120 or 122 and the surgical instrument 210 as indicated on the display unit 225 step 630, and drilling a hole in the bone 100 in alignment with the transverse hole 120 or 122 step 635.

The position and orientation of the surgical instrument 210 relative to the transverse hole 120 or 122 is determined by the transmission of the magnetic signal from the first electromagnetic sensor unit 205 and receiving the signal at the second electromagnetic sensor unit 220. The signals provide the position and location of the electromagnetic sensor units 205 and 220 in relation to one another. The signals are converted into position and orientation data format readable by a programmed computer. The converted data is displayed on the display unit 225, enabling the alignment of the surgical instrument 210 with the transverse hole 120 or 122, by viewing the virtual representation of the position and orientation of the transverse hole 120 or 122 and the surgical instrument 210.

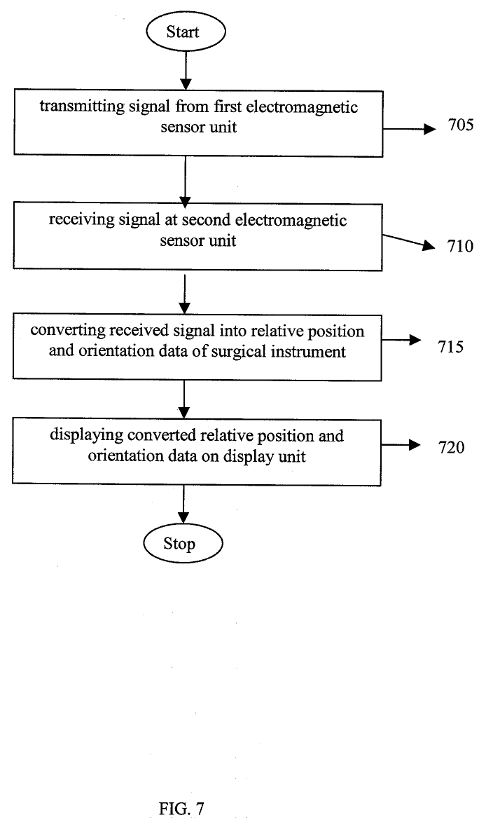

This is further explained in conjunction with FIG. 7. The step 625 of determining the position and orientation of the surgical instrument 210 relative to the transverse hole 120 or 122 comprises steps of transmitting a signal from the first electromagnetic sensor unit 205 step 705, receiving the signal at the second electromagnetic sensor unit 220 step 710, the signal providing information on the position and orientation of the first electromagnetic sensor unit 205 in relation to the electromagnetic drive unit 215, converting received signal into a relative position and orientation data of the surgical instrument 210 step 715 and displaying converted relative position and orientation data on the display unit 225 step 720.

In an exemplary embodiment, the invention provides an apparatus for aligning the surgical instrument 210 with the distal transverse hole 122 of an intramedullary rod 105.

The first electromagnetic sensor unit 205 is inserted into the intramedullary rod 105, down to the level of the interlocking holes, and the electromagnetic drive unit 215 on the surgical instrument 210 energizes the first electromagnetic sensor unit 205 to emit a signal. The second electromagnetic sensor unit 220 positioned external to the patient 305 receives the signal and reports the position of the drill trajectory of the surgical instrument 210 relative to the transverse hole 122 in the intramedullary rod 105.

The positions of the intramedullary rod 105 and the surgical instrument 210 are determined and graphic representations of the intramedullary rod 105 and the surgical instrument 210 are superimposed on the display unit 225. This allows the surgeon to view, in real time, the position of the intramedullary rod 105 and the surgical instrument 210 with respect to at least one image of the patient 305, enabling the surgeon to accurately predict the trajectory of a guide pin that passes through the bore of the surgical instrument 210. The guide pin, once inserted, may be used as a reference for the insertion of the intramedullary rod 105.

In yet another embodiment, a support member (not shown) can be used to releasably support the proximal end 110 of the intramedullary rod 105. A third electromagnetic sensor unit (not shown) can be positioned within the support member (not shown) and can be configured to communicate with the electromagnetic drive unit 215 in the surgical instrument 210 to enable the calculation of the relative locations of the transverse holes 120 and 122 and the surgical instrument 210.

While the above description relates to the placement of the interlocking screws 126 and 128 in the intramedullary rod 105 placed in long bones, persons skilled in the art will recognize the applicability of this invention to other devices in other locations of the body such as the insertion of screws into other surgical implantable devices.

The principal advantages of the invention are: distal targeting of locking screws is accomplished completely without use of C-arm x-ray machines, thereby, eliminating the hazards of radiation exposure to the patient, the risk of radiation exposure to the surgeon, and the need for these costly machines in operating rooms where they are not otherwise available. Accuracy of alignment is greater than that possible with C-arms because mis-alignment is measured by sensitive electronic devices and displayed with visual magnification. Visual indication of alignment is provided continuously, so accuracy of hole drilling does not depend on a surgeon's ability to blindly hold a guide steady while drilling, as required in other freehand procedures that utilize C-arms. The manual dexterity and experience required for surgeons to confidently perform distal targeting are greatly reduced. Surgeons may work in a comfortable and therefore more effective position, rather than from an awkward side position required to avoid the placing of hands into an x-ray beam, as in the use of radiolucent drills. The instrumentation reduces the time required to complete distal screw placement and thereby increases the cost effectiveness of operating rooms.

In addition to the primary application of orthopedic drilling for placement of transverse locking screws in intramedullary rods, there are various possible applications such as locating the position and orientation of other medical devices such as a catheter or a guide wire and as well as in other fields of human and veterinary medical practice.

There are also various possible applications of the instrumentation in non-medical fields: In the construction trades and in certain manufacturing processes, it is desired to accurately cut or drill holes from one side of a wall or sheet of material, when the position of the cut or the hole is known only on the reverse side, and it is either impossible or difficult to transfer the required location to the side from which the work can be done. The device for locating the transverse hole can be adapted to readily perform the desired alignment of cutting or drilling tools for this purpose.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for locating a transverse hole disposed at a predetermined location in a surgical implant inserted into a fractured bone of a patient and for drilling the fractured bone coaxially with the transverse hole, the system comprising:
a first electromagnetic sensor unit coupled to the surgical implant;
a surgical instrument for drilling a hole through the fractured bone coaxially with the transverse hole;
an electromagnetic drive unit coupled to the surgical instrument for energizing the first electromagnetic sensor unit to transmit a signal;
a second electromagnetic sensor unit configured for receiving the signal; and
a display unit coupled to the first and second electromagnetic sensor units for converting the signal into visual signals enabling a user to change the position of the surgical instrument to align the surgical instrument with the transverse hole of the surgical implant.

2. The system of claim 1, wherein the first electromagnetic sensor unit is coupled to a distal end of the surgical implant.

3. The system of claim 2, wherein the first electromagnetic sensor unit is placed within the transverse hole of the surgical implant.

4. The system of claim 1, wherein the surgical implant is made of titanium.

5. The system of claim 1, wherein the electromagnetic drive unit comprises at least one transmitter coil capable of generating flux for energizing the first electromagnetic sensor unit.

6. The system of claim 5, wherein the first electromagnetic sensor unit comprises at least one transceiver coil capable of emitting a signal responsive to the flux received from the electromagnetic drive unit, such that the signal is zero when the axis of the first electromagnetic sensor unit is coplanar with the axis of the electromagnetic drive unit.

7. The system of claim 6, wherein the second electromagnetic sensor unit comprises at least one receiver coil capable of receiving the signal emitted by the first electromagnetic sensor unit.

8. The system of claim 1, wherein the surgical implant is an intramedullary rod.

9. The system of claim 1, wherein the surgical instrument is a surgical drill.

10. A method of aligning a surgical instrument with a transverse hole of a surgical implant, the surgical implant insertable into a patient's bone and having a hollow body portion, the method comprising:
inserting a first electromagnetic sensor unit capable of emitting a signal within the hollow body portion of the surgical implant such that the signal passes through the transverse hole of the surgical implant;
exciting the first electromagnetic sensor unit with an electromagnetic drive unit to cause the first electromagnetic sensor unit to emit the signal, the electromagnetic drive unit coupled to the surgical instrument;
tracking the signal emitted by the first electromagnetic sensor unit using a second electromagnetic sensor unit, the signal comprising position and orientation information of the transverse hole of the surgical implant;
displaying the position and orientation of the transverse hole of the surgical implant;
tracking the position and trajectory of the surgical instrument;
displaying the position and trajectory of the surgical instrument;
superimposing the position and orientation of the transverse hole of the surgical implant and the position and trajectory of the surgical instrument on at least one image of the patient; and
aligning the surgical instrument with the position and orientation of the transverse hole.

11. The method of claim 10, wherein the electromagnetic drive unit is placed external to the patient.

12. The method of claim 10, wherein the second electromagnetic sensor unit is placed external to the patient.

13. The method of claim 10, wherein the image of the patient is obtained using an imaging device.

14. A method of securing a surgical implant into a bone of a patient, the method comprising:
placing a first electromagnetic sensor unit in the surgical implant, the surgical implant having at least one transverse hole for receiving a connector to secure the surgical implant to the bone;
inserting the surgical implant into the bone;
placing a second electromagnetic sensor unit external to the body of the patient;
placing an electromagnetic drive unit carried in a fixed position by a surgical instrument;
determining the position and orientation of the surgical instrument relative to the transverse hole;
aligning the surgical instrument with the transverse hole by viewing a virtual representation of the position and orientation of the transverse hole and the surgical instrument as indicated on a display unit, and drilling a hole in the bone, in alignment with the transverse hole.

15. The method of claim 14, wherein the connector is a locking screw.

16. The method of claim 14, wherein the step of determining the position and orientation of the surgical instrument relative to the transverse hole comprises steps of:
transmitting a signal from the first electromagnetic sensor unit;
receiving the signal at the second electromagnetic sensor unit, the signal comprising information on position and orientation of the first electromagnetic sensor unit in relation to the electromagnetic drive unit;
converting received signal into a relative position and orientation data of the surgical instrument; and
displaying converted relative position and orientation data on the display unit.

17. The method of claim 14, wherein the surgical implant has a proximal end and a distal end.

18. The method of claim 17, wherein the first electromagnetic sensor unit is coupled to the distal end of the surgical implant.

19. The method of claim 18, wherein the first electromagnetic sensor unit is placed within the transverse hole of the surgical implant.

20. The method of claim 14, wherein the electromagnetic drive unit comprises at least one transmitter coil capable of generating flux for energizing the first electromagnetic sensor unit.

21. The method of claim 20, wherein the first electromagnetic sensor unit comprises at least one transceiver coil capable of emitting a signal responsive to the flux received from the electromagnetic drive unit, such that the signal is zero when the axis of the first electromagnetic sensor unit is coplanar with the axis of the electromagnetic drive unit.

22. The method of claim 21, wherein the second electromagnetic sensor unit comprises at least one receiver coil capable of receiving the signal emitted by the first electromagnetic sensor unit.

23. The method of claim 14, wherein the surgical implant is an intramedullary rod.

24. The method of claim 14, wherein the surgical instrument is a surgical drill.
